(19) United States
(12) Patent Application Publication
(10) Pub. No.: US 2011/0070174 A1
(43) Pub. Date: Mar. 24, 2011

(54) Benzoyl Peroxide and Sunscreen Agent Combination

(75) Inventors: Bhiku Patel, Chandler, AZ (US); D. Craig Woodward, Plano, TX (US); Philip J. Gordon, Plano, TX (US)

(73) Assignee: PruGen IP Holdings, Inc.

(21) Appl. No.: 12/563,623

(22) Filed: Sep. 21, 2009

(51) Int. Cl. A61K 8/18; A61Q 17/04

(52) U.S. Cl. 424/59

(57) ABSTRACT

A topical treatment for acne vulgaris comprising benzoyl peroxide and a sunscreen base in a stable compound and methods of manufacture and use. The finished compound being in cream, lotion, gel or other topical formulations.

### I. TECHNICAL FIELD

The present invention relates to a pharmaceutical composition for the treatment of certain skin disorders and more specifically to a stable pharmaceutical combination of benzoyl peroxide and sunscreen for topical application and treatment of skin disorders that benefit from such a combination, particularly acne vulgaris.

### II. BACKGROUND OF THE INVENTION AND PRIOR ART

Skin disorders are pervasive throughout the World. Of particular significance is acne vulgaris, a common skin disorder that affects millions of people worldwide. While it can effect individuals of any age it most common during the teenage years. Several pharmaceutical agents are available to treat acne vulgaris. Among those agents is benzoyl peroxide. It has been used for decades and is considered well tolerated and trusted by physicians. Benzoyl peroxide (also referred to as “BPO” throughout) is a keratolytic agent that increases skin turnover and clears pores. Benzoyl peroxide is typically found in gel or cream form in concentrations of 2.0% to 10.0% and is applied directly to affected areas. Once in contact with the skin, benzoyl peroxide quickly breaks down into benzoic acid and oxygen. An important side effect of benzoyl peroxide is that it can make skin more sensitive to sunlight (“photosensitivity”).

At the same time, it is well established that sun exposure increases the risk of certain skin disorders, principle among them being skin cancer. The additive effect of benzoyl peroxide induced photosensitivity with the known adverse effects of sun exposure make the use of a sun protectant all the more important (the phrases sun protectant, skin protectant, sun block, and sunscreen are used interchangeably throughout and are to be broadly understood to include all manner of applied agents employed to protect the skin from sun exposure). Accordingly, for those individuals who do go into the sun, the use of sunscreens is advocated at all times and in particular it is recommended by health care providers that skin protection is used by those being treated with benzoyl peroxide. Despite this recommendation, it is known that compliance is minimal.

As a result, several attempts to reduce BPO induced photosensitivity have been made. However, until the present invention, combining BPO with a sun protectant has rendered inadequate results for the reason that BPO in the presence of a sunscreen agent creates an unstable compound in which the benzoyl peroxide degrades thereby rendering the composition useless. In fact, the prior art generally teaches away from the use of BPO with a sunscreen for this reason, only employing benzoyl peroxide with either one or more stable agents, such as clindomycin, or filtering/buffering agents that increase BPO stability but decrease its efficacy. Significant also in the prior art is that it generally focuses on the BPO and how to bind it so that it does not degrade when mixed with a sunscreen agent.

There is need, therefore, for a product that combines the known effective properties of benzoyl peroxide with a sunscreen agent in a stable combination that does not require BPO filtering or buffering agents. Such a product would focus on creating a sunscreen agent that is formulated in such a way as to decrease the rate of degradation of benzoyl peroxide when the two agents are mixed together. The present invention presents such a solution.

### III. OBJECTS AND ADVANTAGES OF THE PRESENT INVENTION

It is an object of the present invention to create a stable drug combination that combines benzoyl peroxide and a sunscreen agent.

It is a further object of the invention to provide such a combination that does not require the addition of additional medicinal ingredients or filtering or buffering agents to maintain efficacy or stabilize benzoyl peroxide.

It is further an object of the present invention to create a stable drug combination that effectively treats acne vulgaris while simultaneously provides acceptable skin protection from the Sun.

The advantages offered by the present invention include but are not limited to effectively treating skin diseases and disorders in humans through the use of a heretofore unknown stable combination of benzoyl peroxide and a sunscreen agent which enables effective use of benzoyl peroxide while simultaneously protecting the skin from its harmful effects through the application of a sunscreen agent simultaneously with benzoyl peroxide.

### IV. SUMMARY OF THE INVENTION

The present invention comprises a combination of benzoyl peroxide and at least one sunscreen compound for the treatment of skin disorders, particularly acne vulgaris. The inventive formula is manufactured in phases thereby creating a final product in which benzoyl peroxide remains stable in the presence of a sun block agent. The invention is formulated for topical applications and is suitable for gel, cream, ointment, foam, and other formulations.

The preferred embodiment comprises a six-phase process. The first five phases are each batched separately and mixed to create a homogenous mixture. The resulting five-phase mixture is a suncreen preparation that is an oil-in-water emulsion That mixture is then mixed with phase six, benzoyl peroxide in a ratio to reach the desired BPO concentration. For example, a 6 to 1 ratio will yield 6% Benzoyl Peroxide concentration.

The concentration range for the benzoyl peroxide is about 2% to about10%, and the concentration range for sunscreen is agent dependant upon and in an amount sufficient to reach the desired SPF level. In the preferred embodiment, the benzoyl peroxide is at a concentration of about 3% to about 6%. The sunscreen agent in the preferred embodiment is a combination of titanium dioxide, octocrylene, and zinc oxide. But, of course, any acceptable sunscreen agent can be used, singly or in combination, in the inventive formulation to reach any desired BPO concentration.

There has been outlined, rather broadly, the more important features of the invention in order that the detailed description thereof that follows may be better understood, and in order that the present contribution to the art may be better appreciated. There are, of course, additional features of the invention that will be described hereinafter and that will form the subject matter of the invention.

### V. DETAILED DESCRIPTION OF THE PREFERRED EMBODIMENT

Before explaining the preferred embodiment of the present invention in detail, it is to be understood that the present invention is not limited in its application to the details of formulations and arrangements of the components set forth in the following description. The present invention is capable of other embodiments and of being practiced and carried out in various ways. Also, it is to be understood that the phraseology and terminology employed herein are for the purpose of description and should not be regarded as limiting. It is also to be understood that where ranges are provided for the various agents and drug examples, they are approximate ranges and are not to be limiting except where noted otherwise.

Benzoyl peroxide is an organic peroxide consisting of two benzoyl groups (benzoic acid with the H of carboxylic acid removed) joined by a peroxide group. Accepted condensed formulas include C₆H₅—COO—OOC—C₆H₅, PhCO—O—O—COPh, and (PhCO)²O₂. Benzoyl peroxide is used as a radical initiator. Once applied to the skin it quickly degrades into benzoic acid and oxygen. For commercial purposes, BPO comes as a 38% to 45% slurry (particles in suspension).

As an acne treatment, benzoyl peroxide is typically placed over the affected areas in concentrations of 2.0% up to 10%. Benzoyl peroxide works as a keratolytic peeling agent, increasing skin turnover and clearing pores. It is usually applied more than once a day and is found in various forms including cream, gels, lotions, and ointments.

A known effect of BPO is that it makes skin more susceptible to the sun and its deleterious effects. Accordingly, the use of BPO in conjunction with a sunscreen screen is desirable. However, acceptable sun screen agents, when in combination with BPO create an environment in which the BPO degrades almost immediately, thereby rendering the BPO ineffective in providing therapeutic relief.

Prior art attempts to overcome this problem have included the use of filtering agents that bind BPO, thereby slowing its degradation in the presence of a sun screen agent. However, these solutions render the BPO less available overall since it is bound. Other attempts have included the use of antibiotics, such as clindamycin, in conjunction with the BPO. This results in some slowing of the BPO but necessarily requires the use of an additional medicinal agent to do so; this is not always desirable.

The inventors have discovered, however, that if a base solution containing sunscreen is prepared in phases before adding BPO, the resulting combination results in a stable formula in which both the sunscreen and BPO remain effective.

For purposes of the inventive formula, any suitable sunscreen agent can be used. Commonly used agents are those currently approved for use in certain prescribed ranges by the Food and Drug Administration, Code of Federal Regulation, Part 21, Section 352.10:
(a) para-aminobenzoic acid (PABA) up to15 percent.
(b) Avobenzone up to 3 percent.
(c) Cinoxate up to 3 percent.
(d) Dioxybenzone up to 3 percent.
(e) Homosalate up to 15 percent.
(f) Menthyl anthranilate up to 5 percent.
(g) Octocrylene up to 10 percent.
(h) Octyl methoxycinnamate up to 7.5 percent.
(i) Octyl salicylate up to 5 percent.
(j) Oxybenzone up to 6 percent.
(k) Padimate O up to 8 percent.
(l) Phenylbenzimidazole sulfonic acid up to 4 percent.
(m) Sulisobenzone up to 10 percent.
(n) Titanium dioxide up to 25 percent.
(o) Trolamine salicylate up to 12 percent.
(p) Zinc oxide up to 25 percent.

The inventive formula comprises, in the preferred embodiment, titanium dioxide, zinc oxide, and octocrylene as the sunscreen agents. This is a unique combination of physical and chemical sunscreen agents. Moreover, octocrylene is the only chemical sunscreen agent that does not penetrate the skin. This can be particularly advantageous in the case of acne vulgaris wherein the skin is compromised. As those skilled in the arts will realize, however, any single sunscreen agent or combination of agents will work.

The preferred embodiment comprises a multi-phase process. In example 1, below, the first five phases, phases A-E, are each batched separately and mixed to create a homogenous mixture. The resulting phase A-E mixture is a sunscreen preparation that is an oil-in-water emulsion. The A-E mixture is then mixed with phase F, benzoyl peroxide, at a 6 to 1 ratio. This ratio will yield 6% Benzoyl Peroxide, and according to the sunscreen base mixture, about 6.0% Octocrylene, about 7.5% Titanium Dioxide, and about 2.5% Zinc Oxide. These sunscreen agent percentages offer an optimal physical and chemical sunscreen base, unique to the field, that provides excellent sun protection while at the same time does not interfer with the effectiveness of the BPO and also does not degrade the BPO as is seen in the prior (see stability discussion below).

### EXAMPLE 1

| Phase | CTFA Name |
|---|---|
| A | DEIONIZED WATER |
| | DISODIUM EDTA |
| | ALLANTOIN |
| | DOCUSATE SODIUM |
| | SODIUM CARBOXYMETHYLCELLULOSE |
| | METHYLPARABEN |
| | SODIUM HYALURONATE |
| B | GLYCERYL STEARATE (AND) PEG-100 STEARATE |
| | DIMETHICONE (350 CTS) |
| | MINERAL OIL |
| | CAPRYLIC/CAPRIC TRIGLYCERIDE |
| | BHT |
| | CETYL ALCOHOL |
| | POLYSORBATE-20 |
| | PROPYLPARABEN |
| | ZINC OXIDE, C12-15 ALKYL BENZOATE |
| | C12-15 ALKYL BENZOATE, TITANIUM DIOXIDE, |
| | POLYHYDROXYSTEARIC ACID, ALUMINIUM STEARATE, |
| | ALUMINA |
| | ETHYLHEXYL METHOXYCINNAMATE |
| C | COLLOIDAL OATMEAL |
| | OAT FLOUR |
| D | 95% SQUALANE, 5% UBIQUINONE Q10 |
| E | GREEN TEA (CAMELLIA SINENSIS) |
| | LEAF EXTRACT |

The sunscreen base, phases A-E, are combined and then mixed with Phase F, benzoyl peroxide. For example, to reach a 6% benzoyl peroxide concentration, the sunscreen base is mixed with BPO at a 6 to 1 ratio, which creates a stable combination, as described in more detail below.

Other non-limiting examples of mixing the sunscreen base are presented in examples 2 through 6 below. Each example provides the potential phase mixing of the sunscreen base, which is then, once the sunscreen base is made, mixed with BPO in a ratio to reach the desired BPO concentration. Again, the range is normally between about 2% and 10%, with the preferred embodiment being between #5 and 6%; although the range of BPO concentration is not critical to the invention.

### EXAMPLE 2

| Phase | CTFA Name |
|---|---|
| A | DEIONIZED WATER |
| | DISODIUM EDTA |
| | ALLANTOIN |
| | DOCUSATE SODIUM |
| | SODIUM CARBOXYMETHYLCELLULOSE |
| | METHYLPARABEN |
| | SODIUM HYALURONATE |
| B | GLYCERYL STEARATE (AND) PEG-100 STEARATE |
| | DIMETHICONE (350 CTS) |
| | MINERAL OIL |
| | CAPRYLIC/CAPRIC TRIGLYCERIDE |
| | BHT |
| | CETYL ALCOHOL |
| | POLYSORBATE-20 |
| | PROPYLPARABEN |
| | AVOBENZONE |
| | ETHYLHEXYL METHOXYCINNAMATE |
| C | COLLOIDAL OATMEAL |
| | OAT FLOUR |
| D | DEIONIZED WATER |
| | TITANIUM DIOXIDE |
| E | 95% SQUALANE, 5% UBIQUINONE Q10 |
| | GREEN TEA (CAMELLIA SINENSIS) |
| | LEAF EXTRACT |

### EXAMPLE 3

| Phase | CTFA Name |
|---|---|
| A | DEIONIZED WATER |
| | DISODIUM EDTA |
| | ALLANTOIN |
| | DOCUSATE SODIUM |
| | SODIUM CARBOXYMETHYLCELLULOSE |
| | METHYLPARABEN |
| | SODIUM HYALURONATE |
| B | GLYCERYL STEARATE (AND) PEG-100 STEARATE |
| | DIMETHICONE (350 CTS) |
| | MINERAL OIL |
| | CAPRYLIC/CAPRIC TRIGLYCERIDE |
| | BHT |
| | CETYL ALCOHOL |
| | POLYSORBATE-20 |
| | PROPYLPARABEN |
| C | COLLOIDAL OATMEAL |
| | OAT FLOUR |
| D | DEIONIZED WATER |
| | TITANIUM DIOXIDE |
| E | 95% SQUALANE, 5% UBIQUINONE Q10 |
| | GREEN TEA (CAMELLIA SINENSIS) |
| | LEAF EXTRACT |

### EXAMPLE 4

| Phase | CTFA Name |
|---|---|
| A | DEIONIZED WATER |
| | DISODIUM EDTA |
| | ALLANTOIN |
| | DOCUSATE SODIUM |
| | SODIUM CARBOXYMETHYLCELLULOSE |
| | METHYLPARABEN |
| | SODIUM HYALURONATE |
| B | GLYCERYL STEARATE (AND) PEG-100 STEARATE |
| | DIMETHICONE (350 CTS) |
| | MINERAL OIL |
| | CAPRYLIC/CAPRIC TRIGLYCERIDE |
| | BHT |
| | CETYL ALCOHOL |
| | POLYSORBATE-20 |
| | PROPYLPARABEN |
| C | COLLOIDAL OATMEAL |
| | OAT FLOUR |
| D | DEIONIZED WATER |
| | TITANIUM DIOXIDE |
| E | 95% SQUALANE, 5% UBIQUINONE Q10 |
| | GREEN TEA (CAMELLIA SINENSIS) |
| | LEAF EXTRACT |

### EXAMPLE 5

| Phase | CTFA Name |
|---|---|
| A | DEIONIZED WATER |
| | DISODIUM EDTA |
| | ALLANTOIN |
| | DOCUSATE SODIUM |
| | SODIUM CARBOXYMETHYLCELLULOSE |
| | METHYLPARABEN |
| | SODIUM HYALURONATE |
| B | GLYCERYL STEARATE (AND) PEG-100 STEARATE |
| | DIMETHICONE (350 CTS) |
| | MINERAL OIL |
| | CAPRYLIC/CAPRIC TRIGLYCERIDE BHT |
| | CETYL ALCOHOL |
| | POLYSORBATE-20 |
| | PROPYLPARABEN |
| | AVOBENZONE |
| | ETHYLHEXYL METHOXYCINNAMATE |
| C | COLLOIDAL OATMEAL |
| | OAT FLOUR |
| D | DEIONIZED WATER |
| | TITANIUM DIOXIDE |
| E | 95% SQUALANE, 5% UBIQUINONE Q10 |
| | GREEN TEA (CAMELLIA SINENSIS) |
| | LEAF EXTRACT |

### EXAMPLE 6

| Phase | CTFA Name |
|---|---|
| A | DEIONIZED WATER |
| | DISODIUM EDTA |
| | ALLANTOIN |
| | DOCUSATE SODIUM |
| | METHYLPARABEN |
| B | SODIUM CARBOXYMETHYLCELLULOSE |
| | SODIUM HYALURONATE |
| C | COLLOIDAL OATMEAL |
| | OAT FLOUR |
| D | GLYCERYL STEARATE (AND) PEG-100 STEARATE |
| | DIMETHICONE (350 CTS) |
| | MINERAL OIL |
| | CAPRYLIC/CAPRIC TRIGLYCERIDE |
| | BHT |
| | CETYL ALCOHOL |
| | POLYSORBATE-20 |
| | PROPYLPARABEN |
| | OCTOCRYLENE |
| E | C12-15 ALKYL BENZOATE, TITANIUM DIOXIDE, |
| | POLYHYDROXYSTEARIC ACID, |
| | ALUMINIUM STEARATE, ALUMINA |
| | ZINC OXIDE, C12-15 ALKYL BENZOATE |
| F | 95% SQUALANE, 5% UBIQUINONE Q10 |
| | GREEN TEA (CAMELLIA SINENSIS) |
| | LEAF EXTRACT |
| G | CHAMIMILE TEA FRAGRANCE |
| H | PHENOXYETHANOL |

Example six illustrates that the inventive formula is not limited to a five phase sunscreen base process. Here the sunscreen base is created in an eight-phase process. As those skilled in the arts will understand, the scope and spirit of the invention is maintained, with the breaking up or condensing of phases having no significant impact on the overall invention of creating a stable sunscreen base having no filters or additional medicinal agents, which can then be mixed with benzoyl peroxide. To be clear, the phases are actually batch groups in which the ingredients of each phase is mixed together. Once each group is mixed into a homogeneous phase, the phases are mixed together (before adding the BPO) until a homogeneous sunscreen base is produced. Only then is the BPO added.

Using the above formulas, when mixed with BPO results in a stable BPO/sunscreen combination having a pH of between about 4 and about 8, with the preferred embodiment having a pH of 7.5. pH can be adjusted, if necessary, using commonly available agents. The inventors have found that citric acid and lactic acid work best. The addition of these agents does not affect the efficacy or stability of the BPO or sunscreen base.

The stability provided by using the above formula and mixing process has heretofore been unmatched. Referring to the tables below, in one-month repeatable studies, each of the sunscreen base and the benzoyl peroxide were stable for at least one year. After mixing the agents together, in multiple one-month room temperature studies, the BPO, at various percentages, remained at full potency, as did the various tested sunscreen agents.

STABILITY STUDY 1

| | % INITIAL | 1 MONTH-RT |
|---|---|---|
| BPO | 7.16 (100%) | 7.76 (108%) |
| Octocrylene | 6.33 (100%) | 6.42 (101%) |
| TiO2 | 7.34 (100%) | 7.01 (96%) |
| Zinc Oxide | 2.74 (100%) | 2.75 (100%) |

STABILITY STUDY 2

| | % INITIAL | 1 MONTH-RT |
|---|---|---|
| BPO | 8.55 (100%) | 8.07 (94%) |
| Avobenzone | 3.23 (100%) | 2.78 (86%) |
| OMC | 7.66 (100%) | 7.71 (100%) |

STABILITY STUDY 3

| | % INITIAL | 1 MONTH-RT |
|---|---|---|
| BPO | 6.02 (100%) | 6.48 (107%) |
| Avobenzone | 2.94 (100%) | 2.69 (91.5%) |
| OMC | 7.01 (100%) | 6.74 (96%) |

STABILITY STUDY 4

| | % INITIAL | 1 MONTH-RT |
|---|---|---|
| BPO | 7.16 (100%) | 7.76 (108%) |
| Octocrylene | 6.33 (100%) | 6.42 (101%) |
| TiO2 | 7.34 (100% | 7.01 (96%) |
| Zinc Oxide | 2.74 (100% | 2.75 (100%) |

Owing to the unique nature of the inventive formula and mixing process, the finished BPO/sunscreen mixture can be in cream, lotion, or gel formulations. Moreover, it is contemplated that the final mixture can be sold in a tube in either OTC or Rx formulations. The inventors also contemplate that in order to maximize efficacy, the final mixing of the sunscreen base and the BPO can be done by a physician, pharmacist, or even the patient.

It is to be understood, however, that even though numerous characteristics and advantages of the preferred and alternative embodiments have been set forth in the foregoing description, together with details of the structure and function of the embodiments, the disclosure is illustrative only, and changes may be made in detail within the principles of the invention to the full extent indicated by the broad general meaning of the terms in which the appended claims are expressed.

We claim:

1. A pharmaceutical composition comprising a benzoyl peroxide in combination with one or more sunscreen agents.

2. The pharmaceutical composition of claim 1 wherein the benzoyl peroxide is in the range of about 2% to about 10%.

3. The pharmaceutical composition of claim 1 wherein the benzoyl peroxide is 6%.

4. The pharmaceutical composition of claim 1 wherein the benzoyl peroxide is 3%.

5. The pharmaceutical composition of claim 1 wherein the sunscreen agents are singly or in combination selected from the group comprising para aminobenzoic acid (PABA), Avobenzone, inoxate, Dioxybenzone, Homosalate, Menthyl anthranilate, Octyl methoxycinnamate, Octyl salicylate, Oxybenzone, Padimate O, Phenylbenzimidazole sulfonic acid, Titanium dioxide, Trolamine salicylate, and Zinc oxide.

6. The sunscreen agent of claim 5 being a combination of octocrylene, titanium oxide, and zinc oxide.

7. The pharmaceutical composition of claim 1 further including a soothing agent.

8. The soothing agent of claim 6 comprising oatmeal.

9. The pharmaceutical composition of claim 1 further including a fragrance.

10. The pharmaceutical composition of claim 1 having a pH of between about 4 and about 8.

11. The pH of claim 10 being adjustable by use of citric acid or lactic acid.

12. The pharmaceutical composition of claim 10 being stable for a period of not less than thirty days.

13. The pharmaceutical composition of claim 10 being stable for at least ninety days.

14. A topical pharmaceutical composition comprising benzoyl peroxide in a range of about 2% to about 10% in combination with one or more sunscreen agents wherein the combination is stable for not less than thirty days.

15. The topical pharmaceutical composition of claim 14 being stable for at least ninety days.

16. The pharmaceutical composition of claim 14 wherein the sunscreen is selected from the group comprising Aminobenzoic acid (PABA), Avobenzone, inoxate, Dioxybenzone, Homosalate, Menthyl anthranilate, Octyl methoxycinnamate, Octyl salicylate, Oxybenzone, Padimate O, Phenylbenzimidazole sulfonic acid, Titanium dioxide, Trolamine salicylate, and Zinc oxide.

17. The sunscreen agent of claim 16 being a combination of octocrylene, titanium oxide, and zinc oxide.

18. The pharmaceutical composition of claim 14 further including a soothing agent.

19. The soothing agent of claim 18 comprising oatmeal.

20. The pharmaceutical composition of claim 9 further including a fragrance.

21. The pharmaceutical composition of claim 14 having a pH of between about 4 and about 8.

22. The pH of claim 21 being adjustable by use of citric acid or lactic acid.

23. The pharmaceutical composition of claim 14 being stable for a period of not less than thirty days.

24. The pharmaceutical composition of claim 14 being stable for at least ninety days.

25. A method a preparing a topical pharmaceutical composition comprising the steps of:
preparing a suncreen base that is an oil-in-water emulsion; and,
mixing benzoyl peroxide into the sunscreen preparation to form a benzoyl peroxide-sunscreen compound.

26. The method of claim 25 further including the step of preparing the sunscreen base in a multiple phase process.

27. The method of claim 26 wherein the multiple phase process includes the steps of mixing sunscreen base ingredients in separate phase batches and then, once each phase is mixed, the individual phase are mixed together, without the addition of benzoyl peroxide, into a homogeneous mixture.

28. The method of claim 27 further including the steps of producing sunscreen base phase comprising

| | |
|---|---|
| A | DEIONIZED WATER |
| | DISODIUM EDTA |
| | ALLANTOIN |
| | DOCUSATE SODIUM |
| | SODIUM CARBOXYMETHYLCELLULOSE |
| | METHYLPARABEN |
| | SODIUM HYALURONATE |
| B | GLYCERYL STEARATE (AND) PEG-100 STEARATE |
| | DIMETHICONE (350 CTS) |
| | MINERAL OIL |
| | CAPRYLIC/CAPRIC TRIGLYCERIDE |
| | BHT |
| | CETYL ALCOHOL |
| | POLYSORBATE-20 |
| | PROPYLPARABEN |
| | ZINC OXIDE, C12-15 ALKYL BENZOATE |
| | C12-15 ALKYL BENZOATE, TITANIUM DIOXIDE, |
| | POLYHYDROXYSTEARIC ACID, |
| | ALUMINIUM STEARATE, ALUMINA |
| | ETHYLHEXYL METHOXYCINNAMATE |
| C | COLLOIDAL OATMEAL |
| | OAT FLOUR |
| D | 95% SQUALANE, 5% UBIQUINONE Q10 |
| E | GREEN TEA (CAMELLIA SINENSIS) |
| | LEAF EXTRACT |

29. The method of claim 27 further including the steps of producing sunscreen base phase comprising

| | |
|---|---|
| A | DEIONIZED WATER |
| | DISODIUM EDTA |
| | ALLANTOIN |
| | DOCUSATE SODIUM |
| | METHYLPARABEN |
| B | SODIUM CARBOXYMETHYLCELLULOSE |
| | SODIUM HYALURONATE |
| C | COLLOIDAL OATMEAL |
| | OAT FLOUR |
| D | GLYCERYL STEARATE (AND) PEG-100 STEARATE |
| | DIMETHICONE (350 CTS) |
| | MINERAL OIL |
| | CAPRYLIC/CAPRIC TRIGLYCERIDE |
| | BHT |
| | CETYL ALCOHOL |
| | POLYSORBATE-20 |
| | PROPYLPARABEN |
| | OCTOCRYLENE |
| E | C12-15 ALKYL BENZOATE, TITANIUM DIOXIDE, |
| | POLYHYDROXYSTEARIC ACID, |
| | ALUMINIUM STEARATE, ALUMINA |
| | ZINC OXIDE, C12-15 ALKYL BENZOATE |
| F | 95% SQUALANE, 5% UBIQUINONE Q10 |
| | GREEN TEA (CAMELLIA SINENSIS) |
| | LEAF EXTRACT |
| G | CHAMIMILE TEA FRAGRANCE |
| H | PHENOXYETHANOL |
